(19) United States
(12) Patent Application Publication
(10) Pub. No.: US 2005/0127103 A1
(43) Pub. Date: Jun. 16, 2005

(54) Dispensing machine

(75) Inventor: John Nicholas Reid, Great Wakering, Essex (GB)

Correspondence Address: SALTAMAR INNOVATIONS, 30 FERN LANE, SOUTH PORTLAND, ME, 04106, US

(73) Assignee: Vallid Limited

(21) Appl. No.: 10/504,234

(22) PCT Filed: Feb. 20, 2002
(86) PCT No.: PCT/GB02/00740

(51) Int. Cl. B67D 5/58

(52) U.S. Cl. 222/190

(57) ABSTRACT

A machine is disclosed for dispensing a comestible product. The machine has a delivery outlet (18) through which the product passes during dispensing operations, and means (22, 26) for sterilising the outlet during periods between dispensing operations by exposing the outlet to a sterilisation fluid containing ozone. The mouth of the outlet (18) includes a non-drip valve (20) and the machine is constructed in a manner to prevent exposure of the product (10) stored in the machine to ozone.

### FIELD OF THE INVENTION

The present invention relates to a machine for dispensing a comestible product.

### BACKGROUND OF THE INVENTION

It has hitherto been difficult to dispense certain products, in particular milk, through a delivery outlet in a dispensing machine because of the risk of contamination of the delivery outlet. Hence, for example, in drinks machines, milk is added to tea and coffee in powder form rather than in liquid form to avoid the risk of such contamination.

U.S. Pat. No. 4,867,052 describes an ice-cream dispensing machine having a delivery outlet through which the ice-cream flows during dispensing operations, and means for exposing both the outlet and the ice-cream storage compartment to ozone to maintain the machine sterile at all times.

Despite the passing of more than ten years since the latter patent was issued and the significant demand for such a machine, automatic ice-cream dispensing machines sterilised by the use of ozone have not appeared in the marketplace. The reason for this is believed to be that exposure to ozone of comestible products, such as milk, that contain proteins and carbohydrates quickly renders the products rancid and unfit for consumption.

### OBJECT OF THE INVENTION

The present invention seeks to provide a dispensing machine that enables a comestible product such as milk to be dispensed safely without the risk of contamination.

### SUMMARY OF THE INVENTION

According to the present invention, there is provided a machine for dispensing a comestible product having a delivery outlet through which the product passes during dispensing operations, and means for sterilising the outlet during periods between dispensing operations by exposing the outlet to a sterilisation fluid containing an oxidising agent, wherein the mouth of the outlet includes a non-drip valve and wherein the machine is constructed in a manner to prevent exposure of the product stored in the machine to the oxidising agent.

For ease of reference, it will be assumed hereinafter is that ozone is the oxidising agent used for sterilisation. Ozone is preferred because it can be locally generated, by means of a spark discharge or a source of ultraviolet light. It would be alternatively possible however to use other oxidising agents, such as chlorine dioxide.

By providing a non-drip valve at the mouth of the delivery outlet, it is possible to ensure that no drips of the product adhere to any part of the delivery outlet to become rancid when the delivery outlet is sterilised with ozone. The ozone is also prevented from reaching the stored product so that the quality of the stored product yet to be dispensed is not adversely affected by the ozone.

The non-drip valve may be of any suitable construction and may for example be a pintle valve as used in motor vehicle fuel injectors. It is preferred however for reasons of simplicity and low cost to use a sphincter valve as already known for example from squeeze bottles used to dispense shampoo. Such a sphincter valve comprises a dished plastics membrane formed with cruciform slits. When the product is not under pressure, the valve lies concave and the sides of the slits seal against one another to prevent leakage of the product. When the product is pressurised on the other hand, the membrane is deflected to be dished convex outwards and now the sides of the slits can separate to allow the product to be dispensed.

To prevent ozone from reaching the stored product, it would be possible to store the product in a sealed flexible bag that is squeezed or collapsed when the product is to be dispensed. Such an approach is not however preferred as it requires special packaging.

To enable the product to be stored in a conventional container, such as a bottle or a carton, it is preferred to provide a supply of sterile ozone-free gas that is introduced into the container to replace the volume of the product drawn from the container during the dispensing operation.

The product is preferably drawn from the container by means of a plastics tube immersed into the product and along which the product advanced by means of a peristaltic pump. Such pumps are well known and used in medical applications where a liquid needs to be pumped while remaining in a sterile environment.

Such a pump also has the advantage of being capable of dispensing accurately metered quantities. The pump is also reversible allowing a low pressure to be applied at the mouth of the delivery outlet at the end of a dispensing operation to prevent drips.

The ozone may be dissolved in a liquid such as water but it is simpler and preferred to administer the ozone in gaseous form. The use of either form of fluid ensures that all exposed parts of the nozzle come into contact with the ozone.

To prevent ozone from being discharged into the atmosphere, it is desirable to provide an active charcoal filter through which all air to be discharged from the machine is passed. The ozone free and sterile exhaust gas from this filter may advantageously be used to occupy the volume of the dispensed product within the product storage container.

### BRIEF DESCRIPTION OF THE DRAWINGS

The invention will now be described further, by way of example, with reference to the accompanying drawings, in which:

FIG. 1 is a schematic block diagram of a machine in accordance with the invention for dispensing milk,

FIG. 2 is a section through the mouth of the delivery outlet showing a sphincter valve, and

FIG. 3 is a view of the sphincter valve of FIG. 2 looking directly into the mouth of the delivery outlet.

### DETAILED DESCRIPTION OF THE PREFERRED EMBODIMENTS

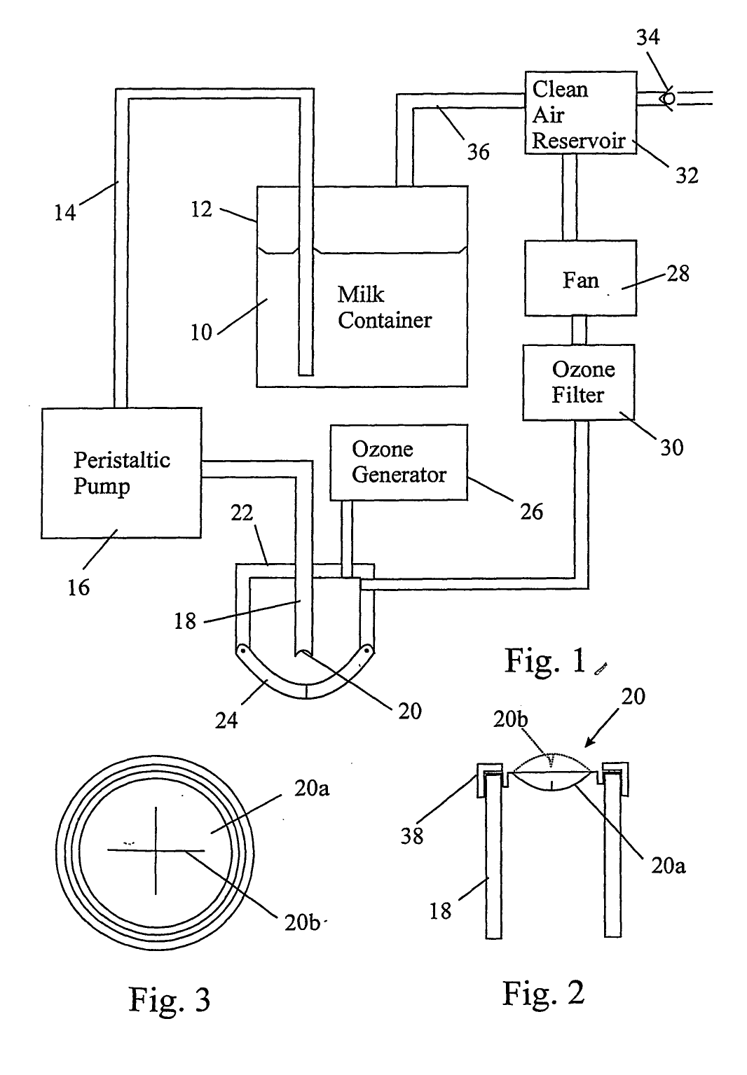

FIG. 1 shows parts of a machine for dispensing milk. The milk 10 is held in an open container 12 which may be a conventional milk bottle or carton. Milk 10 is drawn from the container 12 through a flexible plastic pipe 14 immersed in the milk. The pipe 14 passes through a peristaltic pump 16 which pumps the milk into a delivery outlet 18 terminating at its mouth in a sphincter valve 20 shown more clearly in FIGS. 2 and 3.

The delivery outlet 18 is surrounded by a sterilisation chamber 22 having two hinged doors 24 that open while milk is being dispensed and close around the delivery outlet 18 (as shown) in between dispensing operations.

An ozone generator 26 is provided to produce ozone by means of a spark discharge or by emitting ultraviolet light. Air with a small concentration of ozone is drawn into the sterilisation chamber 22 after each dispensing operation by means of a fan 28. Before reaching the fan, the air passes through an ozone filter 30, which may be a filter containing active charcoal or other suitable reagent such as potassium permanganate, to convert any ozone back into oxygen. The ozone filter 30 ensures that after it has been used to sterilise the delivery outlet 18, the generated ozone is neutralised before reaching any other part of the machine. This is important as ozone would cause damage to wiring insulation, and corrode metal machine components. Ozone would also render any milk that it contacts rancid.

The exhaust gas from the ozone filter 30 passes into a clean air reservoir 32 which is vented to atmosphere through a non-return valve 34 and is connected to the ullage space of the container 12 through a pipe 36. The clean air reservoir 32 should have a variable volume and may for example be constructed as a chamber with collapsible walls or it may include a bellows The sphincter valve 20, as shown in FIGS. 2 and 3, is an inexpensive form of non-drip valve. The valve 20 consists of a membrane that is retained on the end of the delivery outlet 18 by means of a threaded stainless steel collar 38 to allow the membrane to be replaced easily. The membrane has a concave dished portion 20a with slits 20b in the shape of a cross. When the delivery outlet 18 is not under pressure, the membrane adopts the position shown by a solid line in FIG. 2 in which the slits are closed and no milk can leave the outlet. When the outlet 18 is pressurised by the peristaltic pump, the membrane is deflected into the position shown in dotted lines where the sides of the slits separate to allow the milk to be dispensed.

In operation of the machine, when milk is to be dispensed a button is depressed, at which time the ozone generator 26 is switched off. The fan 28 is operated to draw ambient air into the sterilisation chamber and to evacuate ozone containing air already present in the sterilisation chamber. The ozone removed from the sterilisation chamber is converted to oxygen by the filter 30 and the resultant sterile air is blown into the clean air reservoir 32. The latter remains under a pressure slightly higher than atmospheric pressure on account of the pressure drop across the non-return valve 34.

After the sterilisation chamber has been purged of ozone, the doors 24 are opened either mechanically or preferably by means of a motor (not shown). Once the doors 24 have opened, the pump 16 is operated to pump milk 10 out of the container 12 into the delivery outlet 18, the milk being thus dispensed through the sphincter valve 20 which opens automatically in the manner previously described. The volume of the milk drawn from the container 12 is replaced by sterilised air that enters the ullage space of the container 12 from the clean air reservoir 32. Because of the collapsible nature of the reservoir 32, all the air required to replenish the ullage space of the container 12 may be drawn from it even when the fan is not in operation.

At the end of the dispensing operation, the peristaltic pump is stopped and reversed by a small amount. This reversing causes the valve 20 to close abruptly and ensures that the outside of the valve 20 remains drip-free. The doors 24 of the sterilisation chamber 22 are then closed and the ozone generator 26 is switched on for a short time to create ozone that enters the chamber 22 and maintains the outer surfaces of the delivery outlet 18 and the valve 20 sterile between operating cycles.

It is possible to replace the single fan 28 by two separate blowers, one to draw air through the filter 30 and the other located on the intake side of the ozone generator 26 to blow air through the latter. Such an arrangement improves circulation and ensures an adequate flow of air through the ozone generator 26, even in the presence of some leakage of air into the sterilisation chamber 22.

It will be noted ozone is prevented at all times from contacting the milk in the container 12 and from entering the body of the machine where it can cause damage. Instead the ozone is confined to a small region of the machine which can be formed of stainless steel to withstand the very strong oxidation properties of the ozone. The milk may therefore remain in the machine for prolonged lengths of time without deteriorating. Furthermore, because of the use of a non-drip valve at the mouth of the delivery outlet, the invention ensures that the dispensed milk does not come into contact with drips from a previous dispensing cycle that have been rendered rancid by exposure to ozone.

As described, the delivery outlet is sterilised between each dispensing operation but it may be sufficient to effect sterilisation less frequently. For example, it may suffice the carry out a sterilisation cycle once or twice a day regardless of the number of times that the machine is operated to dispense milk.

In an alternative embodiment of the invention, it is possible to replace the traps doors 24 of the sterilisation chamber with a sliding door that is mechanically pushed back by the cup into which milk is to be dispensed, when the user inserts the cup into the machine. In this case, instead of initiating a dispensing operation by depressing a button, the sequence of operations involving the disabling of the ozone generator 26 and the operation of the peristaltic pump 16 can be triggered automatically by movement of the sliding door of the sterilisation chamber. Aside from saving on the cost of a motor to open the doors 24, such an embodiment offers the advantage that the extent to which the sliding door is moved can be used to vary the speed of the peristaltic pump 16, thereby giving the user control over the rate at which milk is delivered. When filling a larger cup, the user would tend naturally insert the cup further into the machine and such action alone will automatically result in an increase in the rate at which the cup is filled.

While the invention has been described by reference to milk, it will be appreciated that it may be used with other comestible products.

1-9. (canceled)

10. A machine for dispensing a comestible product having a delivery outlet through which the product passes during dispensing operations, and a sterilizer for sterilizing the outlet during periods between dispensing operations by exposing the outlet to a sterilization fluid containing an oxidizing agent, wherein the mouth of the outlet includes a drip-free valve and wherein the machine is constructed in a manner to prevent exposure of the product stored in the machine to the oxidizing agent.

11. A machine as claimed in claim 10, wherein the drip-free valve is a sphincter valve (20) which comprises a dished plastics membrane formed with cruciform slits.

12. A machine as claimed in claim 10 wherein the product to be dispensed is stored in a container and wherein the machine further comprises a supply of sterile gas free of the oxidizing agent that is introduced into the ullage space of the container to replace the volume of the product drawn from the container during a dispensing operation.

13. A machine as claimed in claim 12, wherein the product is drawn from the container by means of a tube immersed into the product.

14. A machine as claimed in claim 13, further comprising a peristaltic pump, to advance the product along the tube.

15. A machine as claimed in claim 10, wherein the oxidizing agent is ozone produced within the machine by a generator.

16. A machine as claimed in claim 15 wherein the generator uses a spark discharge or an ultraviolet light, or a combination thereof.

17. A machine as claimed in claim 10, further comprising a filter through which air to be discharged from the machine is passed, to prevent oxidizer from escaping with said air.

18. A machine as claimed in claim 17, wherein the product to be dispensed is stored in a container, and wherein a portion of the air discharged from the filter is introduced into the ullage space of the container to replace the volume of the product drawn from the container during a dispensing operation.

19. A machine as claimed in claim 17, wherein the discharged air from the filter is stored in a variable volume reservoir connected to the ullage space of the product container and from which the stored air is vented to atmosphere through a non-return valve.
